(19) United States
(12) Patent Application Publication
(10) Pub. No.: US 2005/0228452 A1
(43) Pub. Date: Oct. 13, 2005

(54) Steerable catheters and methods for using them

(76) Inventors: Nicholas J. Mourlas, Mountain View, CA (US); Stephen A. Leeflang, Sunnyvale, CA (US); Christian S. Eversull, Palo Alto, CA (US); Christine P. Venture, San Jose, CA (US)

Correspondence Address: COHEN SAKAGUCHI & ENGLISH LLP, 2040 MAIN STREET, 9TH FLOOR, IRVINE, CA, 92614, US

(21) Appl. No.: 11/057,074

(22) Filed: Feb. 11, 2005

Related U.S. Application Data
(60) Provisional application No. 60/544,099, filed on Feb. 11, 2004.
(60) Provisional application No. 60/544,103, filed on Feb. 11, 2004.
(60) Provisional application No. 60/545,865, filed on Feb. 17, 2004.
(60) Provisional application No. 60/549,343, filed on Mar. 1, 2004.
(60) Provisional application No. 60/549,344, filed on Mar. 1, 2004.

(51) Int. Cl. A61N 1/18

(52) U.S. Cl. 607/3; 606/41; 604/510

(57) ABSTRACT

An apparatus for treating tissue includes a flexible catheter including a proximal end, a distal end for introduction into a chamber of a heart, a transparent balloon carried by the distal end, an optical imaging assembly carried by the distal end for imaging tissue structures beyond the distal end through the balloon, and a needle deployable from the tubular member for penetrating the tissue structure to treat tissue. The apparatus may include a source of stems cells or other therapeutic and/or diagnostic agent coupled to the needle, a guide catheter advanceable over the needle for accessing a region beyond the tissue structure penetrated by the needle, and/or an energy probe deployable from the catheter for delivering electrical energy to tissue in the region beyond the tissue structure. The apparatus may be used to deliver stem cells into infracted tissue or for ablating heart tissue, e.g., from a trans-septal approach.

This application claims benefit of provisional application Ser. Nos. 60/544,099 and 60/544,103, filed Feb. 11, 2004, 60/545,865, filed Feb. 17, 2004, and 60/549,343 and 60/549,344, filed Mar. 1, 2004. The entire disclosures of these applications are expressly incorporated herein by reference.

### FIELD OF THE INVENTION

The present invention relates generally to catheters for introduction into body lumens within a patient's body, and, more particularly, to steerable catheters for visualization within a patient's body and/or for accessing body lumens, and to methods for using such catheters.

### BACKGROUND

Minimally invasive procedures have been implemented in a variety of medical settings, e.g., for vascular interventions, such as angioplasty, stenting, embolic protection, electrical heart stimulation, heart mapping and visualization, tissue ablation, and the like. One such procedure involves delivering an electrical lead into a coronary vein of a patient's heart that may be used to electrically stimulate the heart. Another procedure involves delivering an electrode probe into a patient's heart to ablate tissue, e.g., surrounding the pulmonary ostia to treat atrial fibrillation. Steerable catheters have also been suggested to facilitate delivering such devices.

During such procedures, instruments, fluids, and/or medicaments may be delivered within a patient's vasculature using visualization tools, such as x-ray, fluoroscopy, ultrasound imaging, endoscopy, and the like. In many procedures, it may be desirable to deliver instruments through opaque fluids, such as blood, or other materials. Endoscopes have been suggested that include devices for displacing these materials from an optical path, e.g., by introducing a clear fluid from the endoscope in an attempt to clear its field of view. Yet there are still improvements that may be made to such devices.

Accordingly, apparatus and methods for imaging within body lumens and/or for delivering instruments and/or fluids into a patient's body would be useful.

### SUMMARY OF THE INVENTION

The present invention is directed generally to apparatus and methods for accessing body lumens within a patient's body. More particularly, the present invention is directed to steerable catheters for visualization within a patient's body and/or for accessing body lumens, and to methods for using such catheters.

In accordance with one embodiment, an apparatus is provided for treating a condition within a patient's heart that includes a flexible tubular member including a proximal end, a distal end sized for introduction into a body lumen, a substantially transparent expandable member carried by the distal end of the tubular member, an optical imaging assembly carried by the distal end of the tubular member and at least partially surrounded by the expandable member for imaging tissue structures beyond the distal end through the expandable member, and a needle deployable from the tubular member for penetrating a tissue structure to treat tissue.

For example, in one embodiment, the apparatus may include a source of one or more therapeutic and/or diagnostic agents, e.g., stem cells, coupled to the needle, whereby the agent(s) may be delivered through the needle into the tissue structure penetrated by the needle. In another embodiment, the needle may have a length sufficient to penetrate through the tissue structure into a region beyond the tissue structure. In this embodiment, the apparatus may also include a guide catheter advanceable over the needle for accessing the region beyond the tissue structure penetrated by the needle. In addition or alternatively, the distal end of the tubular member may be tapered such the tubular member may be advanced over the needle into the region beyond the tissue structure after the expandable member is collapsed.

Optionally, the apparatus may also include an energy probe or other instrument deployable through the tubular member. For example, the probe may be used for delivering electrical, laser, thermal, or other energy to tissue in the region beyond the tissue structure.

In accordance with another embodiment, a method is provided for delivering one or more therapeutic and/or diagnostic agents into tissue. A distal end of a tubular member may be advanced into a body lumen, and an expandable member on the distal end of the tubular member may be expanded within the body lumen. The expanded expandable member may be directed against a wall of the body lumen, allowing direct visualization or other imaging through the expandable member to observe tissue beyond the expandable member. The tubular member may be manipulated to move the expandable member relative to the wall to identify a desired tissue structure, and one or more agents may be injected from the tubular member into the desired tissue structure once it is identified. In an exemplary embodiment, the desired tissue structure may include infarcted tissue and the agent(s) may include stem cells to enhance regeneration of the infarcted tissue.

In accordance with yet another embodiment, a method is provided for treating tissue within an organ using a tubular member advanced from a body lumen into a first body cavity, e.g., a first chamber of a heart. An expandable member on the distal end of the tubular member may be expanded within the first body cavity, and advanced against a wall of the body cavity, allowing imaging of tissue through the expandable member. The tubular member may be manipulated to move the expandable member relative to the wall to identify a first tissue structure, e.g., fossa ovalis or other structure on a septum between the first body cavity and a second body cavity. A puncture may be created through the first tissue structure into a second body cavity, and a procedure may be performed within the second body cavity via the puncture.

For example, after collapsing the expandable member, the tubular member may be advanced through the puncture into the second body cavity, whereupon the expandable member may be expanded again within the second body cavity to image tissue surrounding the second body cavity. The tubular member may be manipulated to identify a second tissue structure within the second body cavity, e.g., an ostium of a pulmonary vein. The second tissue structure may be treated, e.g., using a probe advanced through the tubular member. In an exemplary embodiment, the probe may be used to deliver electrical energy (or other electromagnetic energy, e.g., laser, radiofrequency (“RF”), or thermal energy) to ablate or otherwise treat the second tissue structure.

Other aspects and features of the present invention will become apparent from consideration of the following description taken in conjunction with the accompanying drawings.

### BRIEF DESCRIPTION OF THE DRAWINGS

FIG. 1 is a side view of an apparatus, including an imaging catheter having a handle on a proximal end, a balloon on a distal end, a syringe for expanding the balloon, and a monitor for displaying images obtained by the catheter through the balloon.

FIG. 2 is a side view of the catheter of the apparatus of FIG. 1.

FIG. 3 is a side view detail of the distal end of the catheter of FIG. 1, with the balloon in an expanded condition.

FIGS. 4A-4E are cross-sectional views of the catheter of FIG. 2 taken along lines 4A-4A, 4B-4B, 4C-4C, 4D-4D, and 4E-4E, respectively.

FIG. 5 is a side view of the handle of the apparatus of FIG. 1.

FIGS. 6A and 6B are cross-sectional perspective and side views, respectively, of the handle of FIG. 5.

FIG. 7 is a schematic showing components of an imaging assembly that may be included with the apparatus of FIG. 1.

FIGS. 8A and 8B are side views of another embodiment of an apparatus including a needle for delivering one or more agents into tissue.

FIGS. 9A-9C are cross-sectional views of a patient's heart, showing a method for introducing an apparatus into a chamber of the heart to deliver one or more agents into heart tissue.

FIGS. 10A-10D are cross-sectional views of a patient's heart, showing a method for introducing an apparatus into a first chamber of the heart to create a puncture through a wall of the heart into a second chamber of the heart.

FIGS. 11A and 11B are cross-sectional views of an embodiment of an imaging apparatus including a catheter having an expandable sheath that provides an expandable accessory lumen.

FIGS. 12A and 12B are cross-sectional views of another embodiment of an imaging apparatus including a catheter having an expandable sheath that provides an expandable accessory lumen.

FIGS. 13A and 13B are cross-sectional views of still another embodiment of an imaging apparatus including a coiled sheath that provides an expandable accessory lumen.

### DETAILED DESCRIPTION OF THE PREFERRED EMBODIMENTS

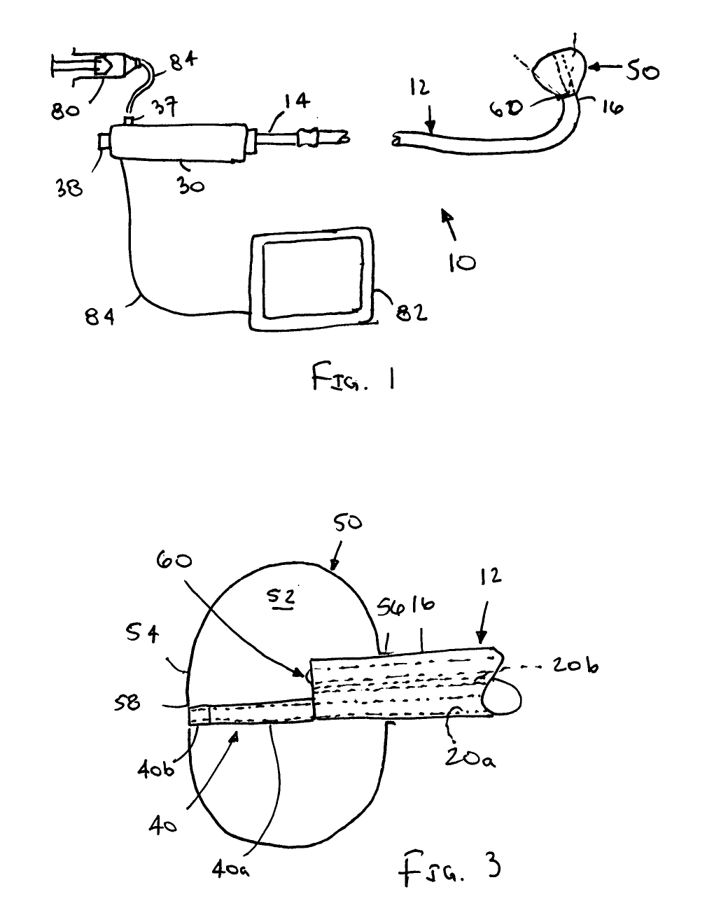

Turning to the drawings, FIG. 1 shows a first embodiment of an apparatus 10 for imaging a body lumen, e.g., for visualizing, accessing, and/or cannulating a body lumen from a body cavity (not shown). As explained further below, the apparatus 10 may be used for imaging a wall of a body lumen, e.g., a right atrium of a heart, e.g., for visualizing, accessing, and/or cannulating a coronary sinus ostium. Alternatively, the apparatus 10 may be used for visualizing, accessing, and/or cannulating other body lumens, e.g., for delivering one or more therapeutic and/or diagnostic agents into tissue, and/or for puncturing through tissue to access a region beyond the punctured tissue. As used herein, “body lumen” may refer to any passage within a patient's body, e.g., an artery, vein, or other blood vessel, or a body cavity, such as a chamber within a patient's heart, e.g., a ventricle or atrium. Although exemplary embodiments are described herein, additional information that may relate to the structure and/or methods for making and/or using the apparatus 10 may also be found in co-pending application Ser. No. 10/447,526, filed May 29, 2003, the entire disclosure of which is expressly incorporated by reference herein.

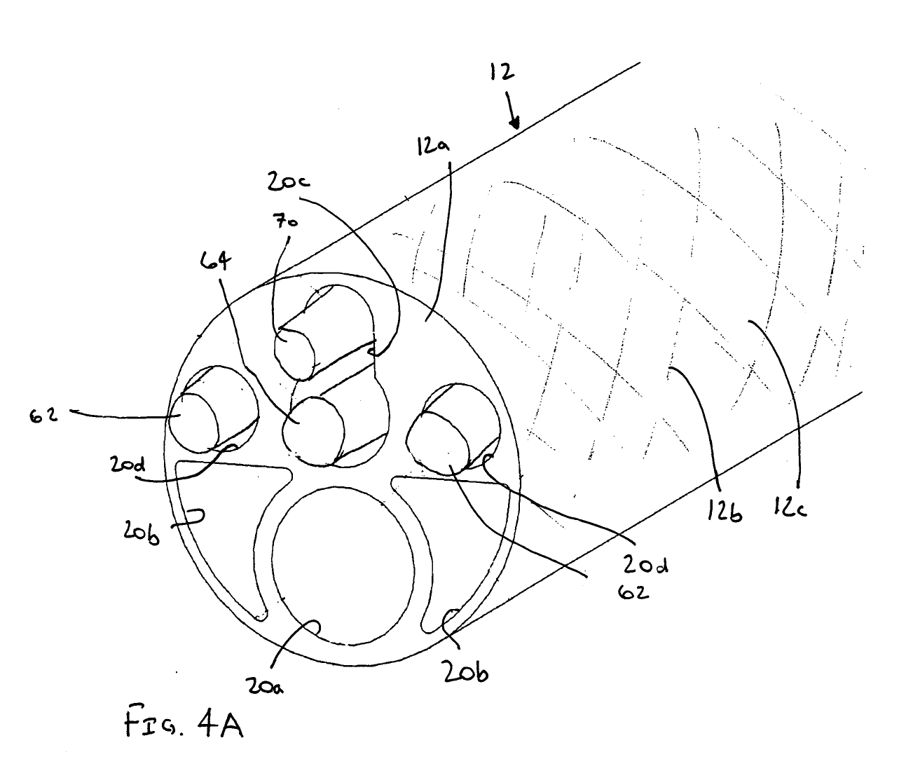

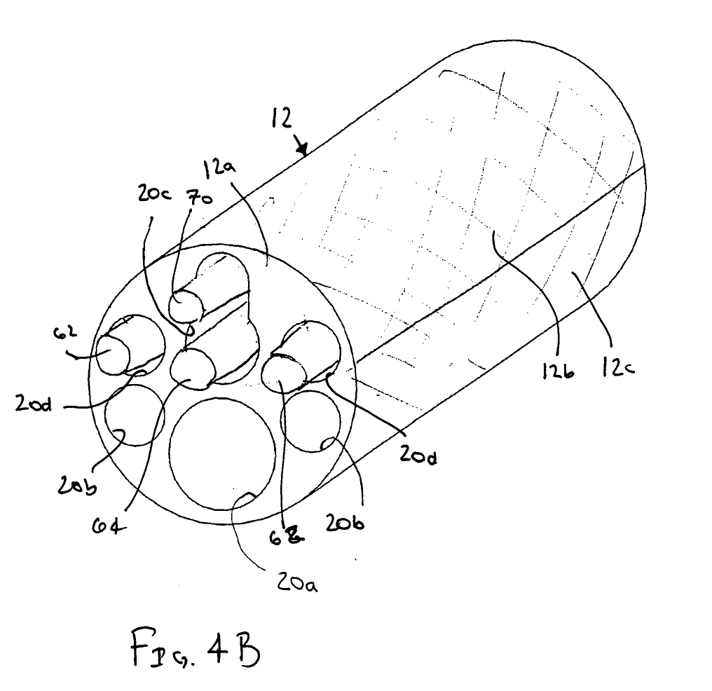

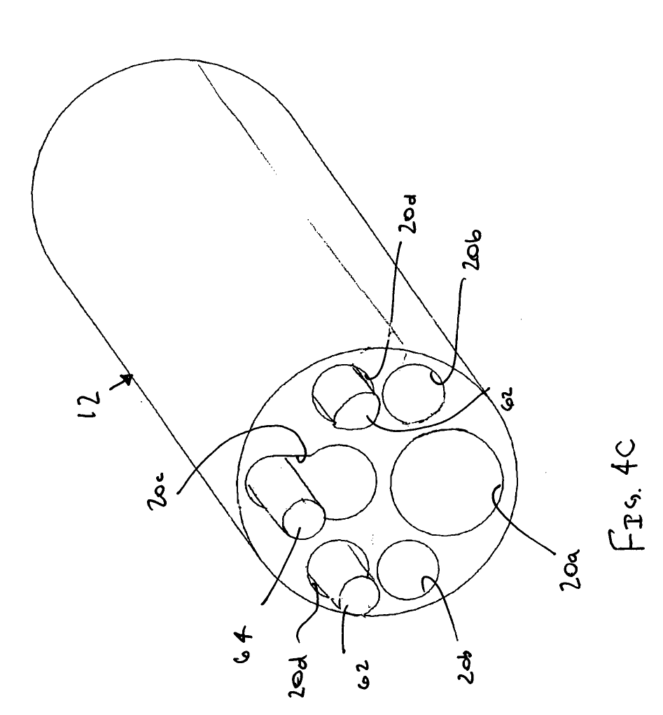

Generally, as shown in FIG. 1, the apparatus 10 includes a catheter or other elongate member 12, including a handle 30 on a proximal end 14 of the catheter 12, and a balloon or other expandable member 50 on a distal end 16 of the catheter 12. An imaging assembly 60 may be provided on or otherwise carried by the catheter 12 for imaging through the balloon 50, e.g. including one or more illumination fibers 62 and/or imaging optical fibers 64 (not shown in FIG. 1, see, e.g., FIGS. 4A-4E) extending through the catheter 12, as described further below. Optionally, the apparatus 10 may include other components, e.g., a syringe or other source of inflation media 80, a monitor or other output device 82, and the like. In additional embodiments, the apparatus 10 may include other devices that may be delivered through, over (e.g., a sheath over the catheter 12), or otherwise advanced from the catheter 12, e.g., a guidewire, a needle, a guide catheter, an energy probe, and the like (not shown), as described further below.

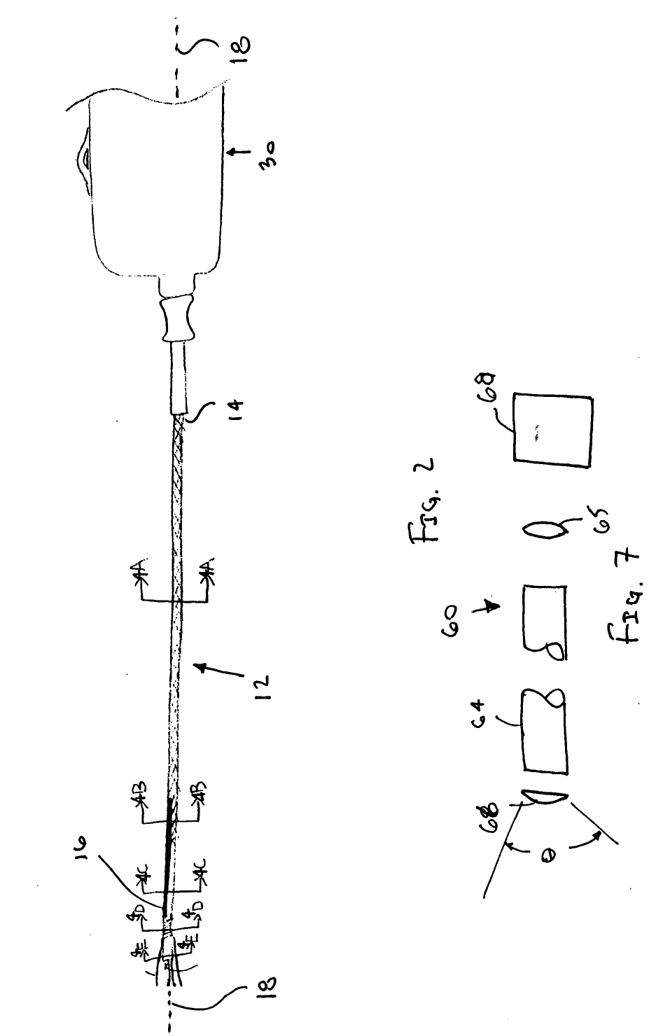

Turning to FIG. 2, the catheter 12 generally is an elongate tubular body including a proximal end 14, a distal end 16 having a size and shape for insertion into a patient's body, and a central longitudinal axis 18 extending between the proximal and distal ends 14, 16. As shown in FIGS. 4A-4E, the catheter 12 may include one or more lumens 20 extending between the proximal and distal ends 14, 16, e.g., an accessory lumen 20a, one or more inflation lumens 20b (two shown), and one or more lumens 20c, 20d for the imaging assembly 60. Optionally, the catheter 12 may include one or more additional lumens (not shown) extending at least partially between the proximal and distal ends 14, 16, e.g., for one or more separate steering elements (not shown). In exemplary embodiments, the catheter 412 may have a diameter between about four and ten French (1.33-3.33 mm), or between about six and eight French (2.00-2.67 mm). In alternative embodiments, the catheter 12 may be used as a guidewire, e.g., having a diameter of not more than about 0.014 inch (0.35 mm) or less.

The catheter 12 may be substantially flexible, semi-rigid, and/or rigid along its length, and may be formed from a variety of materials, including plastic, metal, and/or composite materials. For example, the catheter 12 may be substantially flexible at the distal end 16, e.g., to facilitate steering and/or advancement through tortuous anatomy, and/or may be semi-rigid or rigid at the proximal end 14, e.g., to enhance pushability of the catheter 12 without substantial risk of buckling or kinking. In an exemplary embodiment, the catheter 12 may be formed from PEBAX, which may include a braid or other reinforcement structure therein. For example, as shown in FIGS. 4A and 4B, the catheter 12 may include a plastic core 12a, e.g., polyurethane, extruded or otherwise formed with the lumens 20 therein, over which a braid 12b, e.g., of metal, plastic, or composite fibers, may be disposed. A tube of PET 12c (partially cut away in FIG. 4B) may be disposed around the braid-covered core 12a, and then heat shrunk or otherwise attached to capture and/or secure the braid 12b between the tube 12c and the core 12a. Optionally, an adhesive may be used to bond one or more of the layers 12a-12c of the catheter 12 together.

Optionally, with additional reference to FIG. 3, the catheter 12 may include a tubular extension 40 that extends distally from the distal end 16. The tubular extension 40 has a diameter or other cross-section that is substantially smaller than the catheter 12. In addition, the tubular extension 40 may be offset from or concentric with the central axis 18 of the catheter 12. The tubular extension 40 may facilitate balloon stabilization and/or may maximize a field of view of the imaging assembly 60, as explained further below. The tubular extension 40 may include a section of hypotube or other tubular material, e.g., formed from metal, plastic, or composite materials. In an exemplary embodiment, the tubular extension 40 may include a first section 40a formed from a substantially rigid material, e.g., stainless steel, and a second tip section 40b formed from a flexible material, e.g., PEBAX, to provide a relatively soft and/or atraumatic tip for the apparatus 10. Such a tip section 40b may reduce abrasion or other tissue damage while moving the tubular extension 40 along tissue during use, as explained further below.

The first section 40a may be at least partially inserted into the distal end 16 of the catheter 12, e.g., into the accessory lumen 20a. For example, the material of the distal end 16 may be softened to allow the material to reflow as the first section 40a of the tubular extension is inserted into the accessory lumen 20a. Alternatively, the distal end 16 may include a recess (not shown) sized for receiving a portion of the first section 40a therein. In addition or alternatively, the first section 40a may be attached to the distal end 16 by bonding with adhesive, using mating connectors and/or an interference fit, and the like. The second section 40b may be bonded or otherwise attached to the first section 40a before or after the first section 40a is attached to the distal end 16 of the catheter 12.

Turning to FIGS. 1 and 7, with additional reference to FIGS. 4A-4E, the imaging assembly 60 generally includes an objective lens 66, e.g., a gradient index (“GRIN”) lens, self-oc lens, or other optical imaging element, that is exposed within an interior 52 of the balloon 50 for capturing light images through the balloon 50. The objective lens 66 may be coupled to an optical imaging fiber 64, e.g. a coherent image bundle, that extends between the proximal and distal ends 14, 16 of the catheter 12, e.g., through the lumen 20d, as shown in FIGS. 4A-4E.

In one embodiment, the objective lens 66 may have a diameter similar to the imaging fiber 64, e.g., to simplify bonding and/or alignment, and/or to decrease its overall profile. For example, the objective lens 66 may have a diameter of not more than about three hundred fifty and five hundred microns (350-500 μm). Exemplary lenses may be available from Nippon Sheet Glass (“NSG”) or Grintech.

The objective lens 66 may focus reflected light from images obtained through the balloon 50 onto the face of the imaging fiber 64. The objective lens 66 may have a relatively large numerical aperture (NA), determined by:
NA=sin (Θ/2).
Where Θ is the view angle of the lens 66, as shown in FIG. 7. Alternatively, a wide angle lens may be provided for the objective lens 66 to increase the functional numerical aperture. Optionally, the objective lens 66 may be coated, e.g., to reduce surface reflection and/or otherwise optimize optical properties.

The imaging fiber 64 may include a plurality of individual optical fibers, e.g., between about one thousand and one hundred fifty thousand (1,000-150,000) fibers, or between about three thousand and ten thousand (3,000-10,000) fibers, in order to provide a desired resolution in the images obtained by the optical fiber 64. The material of the imaging fiber 64 may be sufficiently flexible to bend as the catheter 12 bends. Optionally, the imaging fiber 64 may be leached to increase its flexibility.

A device 68 may be coupled or otherwise provided at the proximal end 14 of the apparatus 10 for acquiring, capturing, and/or displaying images transmitted by the imaging fiber 64. As shown in FIG. 7, one or more lenses 65 may be coupled to the fiber bundle 64 for focusing and/or resolving light passing through the imaging fiber 64, e.g., to pass the image to the device 68. The lens 65 may be coupled directly between the imaging fiber 64 and the device 68 or may be spaced apart from one or both the imaging fiber 64 and the device 68. The lens 65 should provide sufficient magnification to prevent substantial loss of resolution, which may depend upon the pixel density of the device 68. For example, a lens 65 having magnification between about 1.3× and 3× may spread a single pixel from the optical fiber 64 onto four or more pixels on the device 68, which may sufficiently reduce resolution loss.

The device 68 may include a CCD, CMOS, and/or other device, known to those skilled in the art, e.g., to digitize or otherwise convert the light images from the imaging fiber 64 into electrical signals that may be transferred to a processor and/or display. The device 68 may be a color device, or may be black and white, which may increase sensitivity. The smaller the pixel size of the device 68, the less magnification that may be needed by the lens 65. In exemplary embodiments, the device 68 may have pixel sizes between about one and ten microns (1-10 μm), or between about two and five microns (2-5 μm).

The device 68 may be coupled to a monitor 82, e.g., by a cable 84, as shown in FIG. 1. In addition or alternatively, a computer or other display or capture devices (not shown) may be coupled to the device 68 to display and/or store the images acquired from the imaging fiber 64. Additional information on capture devices that may be used may be found in application Ser. No. 10/447,526, incorporated by reference herein.

The imaging assembly 60 may also include one or more illumination fibers or light guides 62 carried by the distal end 16 of the catheter 12 for delivering light into the interior 52 and/or through a distal surface 54 of the balloon 50. As shown in FIGS. 4A-4E, a pair of illumination fibers 62 may be provided in the catheter 12. The illumination fibers 62 may be spaced apart from one another, e.g., in separate lumens 20d to minimize shadows, which may be cast by the tubular extension 40. A source of light (not shown) may be coupled to the illumination fiber(s) 62, e.g., via or within the handle 30, for delivering light through the illumination fiber(s) 62 and into the balloon 50.

Optionally, the catheter 12 may be steerable, i.e., the distal end 16 may be controllably deflected transversely relative to the longitudinal axis 18 using one or more pullwires or other steering elements. In the embodiment shown in FIGS. 4A-4E, the imaging fiber 64 may be used for steering the distal end 16 of the catheter 12 in one transverse plane (thereby providing one degree of freedom), as well as for obtaining images through the balloon 50. Alternatively, multiple pullwires (not shown) may be provided for steering the distal end 16 of the catheter 12 in two or more orthogonal planes (thereby providing two or more degrees of freedom).

The imaging fiber 64 (or other pullwire, not shown) may be attached or otherwise fixed relative to the catheter 12 at a location adjacent the distal end 16, offset radially outwardly from a center of modulus of the catheter 12. If the construction of the catheter 12 is substantially uniform about the central axis 18, the center of modulus may correspond substantially to the central axis 18. If the construction of the catheter 12 is asymmetrical about the central axis 18, however, the center of modulus may be offset from the central axis 18 in a predetermined manner. As long as the optical fiber 64 (or other pullwire) is fixed at the distal end offset radially from the center of modulus, a bending moment will result when the imaging fiber 64 is pushed or pulled relative to the catheter 12 to steer the distal end 16.

For example, when the optical fiber 64 is pulled proximally or pushed distally relative to the catheter 12, e.g., from the proximal end 14 of the catheter 12, a bending force may be applied to the distal end 16, causing the distal end 16 to curve or bend transversely relative to the central axis 18. Optionally, as described further below, the degree of steerability of the distal end 16 may be adjustable, e.g., to increase or decrease a radius of curvature of the distal end 16 when the imaging fiber 64 is subjected to a predetermined proximal or distal force. In addition or alternatively, one or more regions of the catheter 12 may be set to be steerable in a predetermined manner.

Turning to FIG. 5, the handle 30 may be an enlarged member coupled to or otherwise provided on the proximal end 14 of the catheter 12. The handle 30 may be contoured or otherwise shaped to facilitate holding the handle 30 and/or otherwise manipulating the catheter 12. The handle 30 may be formed from one or more parts of plastic, metal, or composite material, e.g., by injection molding, and the like, that may be assembled together, e.g., using mating connectors, adhesives, and the like.

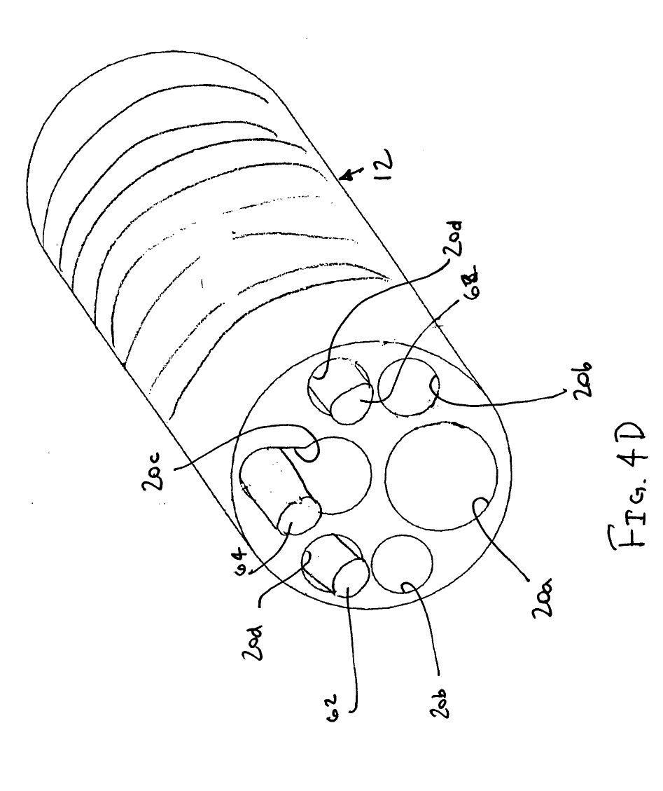

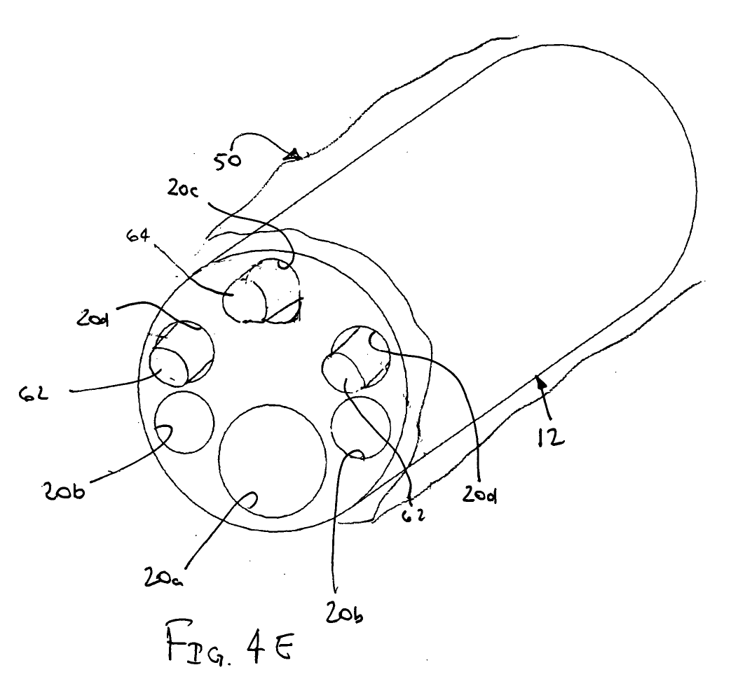

The handle 30 may include one or more steering controls 32, 34 for controlling the ability to steer the distal end 16 of the catheter 12. For example, as shown in FIGS. 6A and 6B, the handle 30 may include an actuator 32 that may be coupled to the optical fiber 64 (not shown in FIGS. 6A-6B) via a linkage 34. The linkage 34 may be pivotally coupled to the handle 30 by a pin 34a such that proximal movement of the actuator 32 causes the linkage 34 to apply a proximal force to the optical fiber 64. The resulting bending moment causes the distal end 16 of the catheter 12 to bend into a curved shape, such as that shown in FIG. 1.

Optionally, the actuator 32 may be biased, e.g., to return the distal end 16 of the catheter 12 to a generally straight configuration when the actuator 32 is released. For example, as shown in FIGS. 6A and 6B, the linkage 34 may be coupled to a resistive mechanism 33 that may allow the actuator 32 to be moved by applying a proximal force to overcome the resistance of the resistive mechanism 33. When a proximal force is removed, e.g., when the actuator 32 is released, the resistive mechanism 33 may return the linkage 34, and consequently the actuator 32 and imaging fiber 64 to a neutral position, thereby substantially straightening the distal end 16 of the catheter 12.

In another embodiment, the resistive mechanism 33 may allow the distal end 16 to maintain a curved configuration once the actuator 32 is moved to steer the distal end 16. As shown in FIGS. 6A and 6B, the resistive mechanism 33 includes a section of tubing 33a coupled to a flexible o-ring 33b that is substantially fixed relative to the handle 30. The o-ring 33b may be secured within a pocket 31 in the handle 30 to prevent the o-ring 33b from moving substantially. The o-ring 33b may be sufficiently flexible to allow the tubing 33a to slide axially through the o-ring 33b when the actuator 32 is pulled, yet may apply a predetermined resistance to such axial movement. Thus, when the actuator 32 is actuated, the resistance of the o-ring 33b may be overcome to cause the distal end 16 of the catheter 12 to curve. When the actuator 32 is released, the o-ring 33b may apply a desired friction against the tubing 33a, thereby preventing the tubing 33a from moving, and consequently maintaining the set curve of the distal end 16. To curve the distal end 16 further or partially or entirely straighten the distal end 16, the actuator 32 may be slid further proximally or distally to overcome the resistance provided by the o-ring 33b. Additional steering elements and structures and methods for using them are disclosed in application Ser. No. 10/447,526, incorporated by reference herein.

In addition, the handle 30 may include a slider 36 for controlling a variable steering radius (“VSR”) mechanism carried by the distal end 16 of the catheter 12. The VSR mechanism may change the radius of curvature of the distal end 16 when the actuator 32 is activated and/or the region of the distal end 16 that is steered, depending upon the relative position of the slider 36. For example, as explained further below, when the slider 36 is in a proximal position, e.g., immediately adjacent the handle 30, the bending moment created when the actuator 32 is activated may be maximized, thereby resulting in a relatively large radius of curvature when the distal end 16 is steered. As the slider 36 is directed distally, the radius of curvature of the distal end 16 may become smaller and more distal.

The handle 30 may also include ports, seals, and/or other connections for connecting other components to the catheter 12 and/or introducing one or more accessories into the catheter 12. For example, as shown in FIG. 5, a port 37 may be provided that communicates with the inflation lumen(s) 20b of the catheter 12 (not shown, see FIGS. 4A-4E). A luer lock or other connector may be provided on the port 37 for temporarily connecting tubing or other fluid-conveying components to the handle 30. As shown in FIG. 1, a syringe or other source of fluid 80, e.g., including saline, carbon dioxide, nitrogen, or air, may be connected to the port 37 via tubing 84 to the inflation lumens 20b of the catheter 12, e.g., for expanding the balloon 50 when fluid is delivered into an interior 52 of the balloon 50. Alternatively, the syringe 80 may be a source of vacuum, e.g., for collapsing the balloon 50 when fluid is evacuated from the interior 52.

Similarly, an access port 38 may be provided that communicates with the accessory lumen 20a of the catheter 12 (also not shown, see FIGS. 4A-4E). Optionally, the access port 38 may include a connector, e.g., a luer lock, and/or one or more seals, e.g., a hemostatic seal, allowing one or more instruments (such as a guidewire, a needle, a guide catheter, and/or an energy probe, not shown) to be inserted through the access port 38 and into the accessory lumen 20a. Alternatively, another source of fluid, e.g., saline, and/or one or more therapeutic or diagnostic agents (not shown), may be connectable via tubing (also not shown) to the accessory lumen 20a, e.g., for delivering fluid beyond the distal end 16 of the catheter 12.

Optionally, the handle 30 may include other components, e.g., a battery or other power source 86, a light source (not shown), e.g., one or more light emitting diodes (“LEDs”) that may be coupled to the illumination fiber(s) 62 for transmitting light beyond the distal end 16 of the catheter 12. In addition, the handle 30 may include a switch 88, e.g., for turning electrical components of the handle 30 on and off, such as the light source.

Returning to FIGS. 1 and 3, a substantially transparent balloon 50 may be provided on the distal end 16 of the catheter 12. The balloon 50 may be expandable from a contracted condition (not shown, see, e.g., FIG. 4E) to an enlarged condition (as shown in FIG. 3), e.g., when fluid is introduced into the interior 52 of the balloon 50. The balloon 50 may be formed from one or more compliant and/or elastomeric materials, such as silicone, latex, or other synthetic or natural elastomers, such as those sold under the trade names Isoprene or Chronoprene. Alternatively, the balloon 50 may be formed from substantially noncompliant material, e.g., polytetrafluoroethylene (PTFE), expanded polytetrafluoroethylene (EPTFE), fluorinated ethylenepropylene (FEP), polyethylene teraphthalate (PET), urethane, olefins, and polyethylene (PE), such that the balloon 50 may expand to a predetermined shape when fully inflated to the enlarged configuration.

In the enlarged condition, the balloon 50 may have a distal surface 54 that is substantially flat or otherwise configured for contacting a wall of a body cavity, such as the right atrium (not shown). The balloon 50 may have a generally spherical shape, a frusto-conical shape, and the like, thereby defining the distal surface 54 beyond the distal end 16 of the catheter 12.

The balloon material may be sufficiently flexible and/or elastic such that the distal surface 54 may conform substantially to the wall of a body cavity. The balloon 50 may also be sufficiently noncompliant to displace blood or other fluid from between the distal surface 54 and the wall of the body cavity to facilitate imaging tissue of the wall through the balloon 50, as explained further below. The balloon 50 may be molded around or within a mold (not shown) having a desired shape for the balloon 50 in the enlarged or contracted condition. Alternatively, the balloon 50 may be formed from one or more panels that may be attached to one another, e.g., using an adhesive (such as an adhesive cured using ultraviolet (“UV”) light), sonic welding, and/or heating, after lapping or butting adjacent panels together.

The balloon 50 may include a proximal end 56 that may be attached to an outer surface of the catheter 12 adjacent the distal end 16, e.g., using an adhesive, heating, sonic welding, an interference fit, and/or an outer sleeve or other wrap (not shown). The distal surface 54 of the balloon 50 may include an opening 58 therein, allowing the balloon 50 to be bonded or otherwise attached to the tubular extension 40 around the opening 58. In one embodiment, the distal surface 54 of the balloon 50 may extend slightly beyond the tip 40b of the tubular extension 40 to enhance the atraumatic character of the apparatus 10 when the balloon 50 is directed against tissue.

As shown in FIG. 3, the interior 52 of the balloon 50 may communicate with the inflation lumen(s) 20b of the catheter 12. Substantially transparent inflation media, e.g., saline, carbon dioxide, nitrogen, air, and the like, may be introduced into the interior 52 of the balloon 50, e.g., from the syringe 80 shown in FIG. 1, to expand the balloon 50 towards the enlarged condition. As used herein, “transparent” refers to any material and/or fluid that may permit sufficient light to pass therethrough in order to identify or otherwise visualize objects through the material and/or fluid. “Light” as used herein may refer to light radiation within the visible spectrum, but may also include other spectra, such as infrared (“IR”) or ultraviolet (“UV”) light.

Alternatively, the balloon 50 may be provided using different configurations, materials, and/or methods, such as those disclosed in co-pending application Ser. No. 10/447,526, incorporated by reference above.

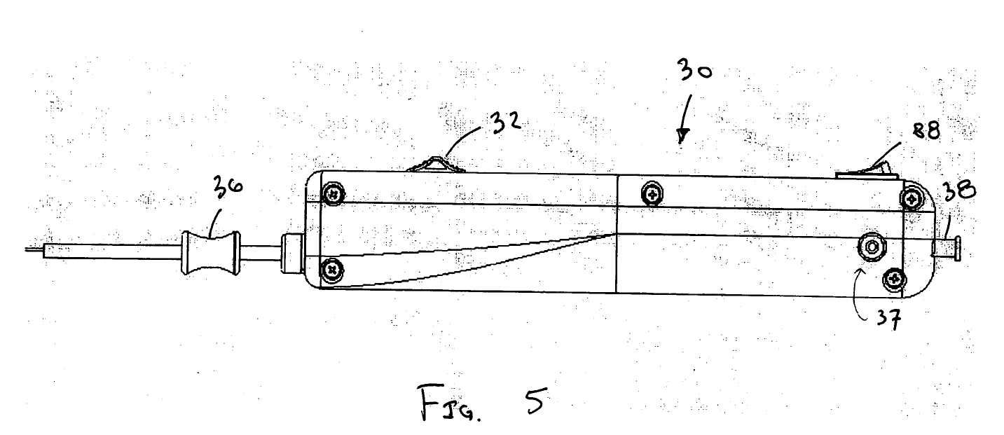

Turning to FIGS. 8A and 8B, the apparatus 10 may include a needle 70 that may be deployable from the distal end 16 of the catheter 12. The needle 70 generally includes a proximal end 72, a distal end 74 sized for insertion into the accessory lumen 20a of the catheter 12 and terminating in a sharpened distal tip 75, and a lumen 76 extending between the proximal and distal ends 72, 74. As shown, the needle 70 is advanceable substantially axially through the accessory lumen 20a of the catheter 12, and consequently, through the tubular extension 40 and beyond the distal surface 54 of the balloon 50. The needle 70 may be formed from stainless steel or other material having sufficient flexibility to be advanced through the catheter 12, e.g., when the catheter 12 has been advanced through tortuous anatomy, yet have sufficient rigidity to be advanced through tissue. The needle 70 may have a single distal opening, or an array of openings (not shown) may be provided in the distal tip 75 for delivering fluid in a desired manner from the distal tip 75. Exemplary configurations of needles and that may be used in association with the apparatus 10 and methods for treating tissue with such needles are disclosed in U.S. Pat. No. 6,283,951, the entire disclosure of which is expressly incorporated by reference herein.

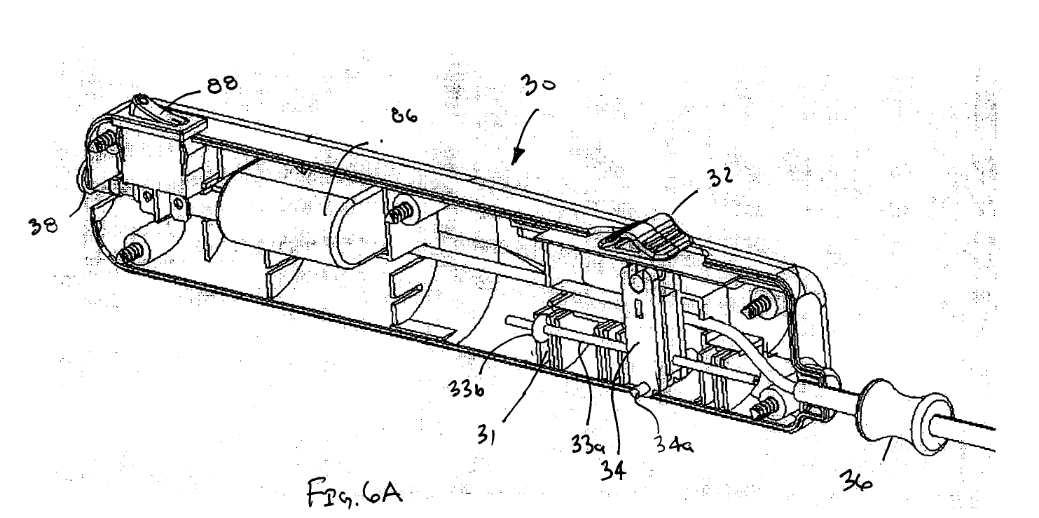

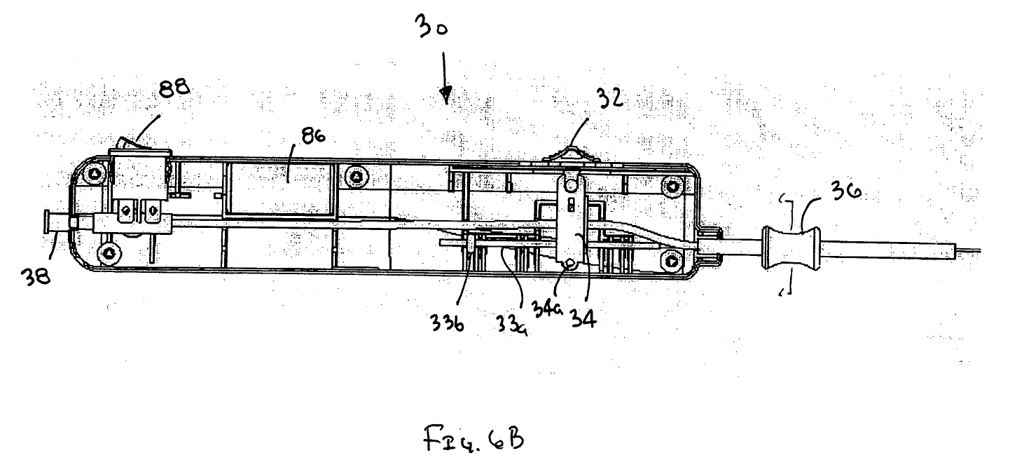

Turning to FIGS. 9A-9C, a method is shown for delivering one or more therapeutic and/or diagnostic agents into tissue within a patient's heart. For example, the apparatus 10 may be used for delivering stem cells into tissue, e.g., that has undergone necrosis after an acute myocardial infarction. It has been found that injecting necrotic tissue with stem cells may restore contractility to large volumes of heart tissue. However, because of the scarcity and cost of stem cells, the stem cells should only be delivered into necrotic tissue and not into otherwise healthy tissue where it is not needed.

The distal end 16 of the apparatus 10 may be introduced into a patient's body using conventional methods used for delivering catheters or other instruments. For example, with the balloon 50 collapsed, the distal end 16 of the catheter 12 may be introduced into a patient's vasculature, e.g., from a percutaneous puncture, e.g., in a peripheral vessel, such as a femoral artery or vein, carotid artery, and the like, depending upon which side of the heart is to be treated. For example, as shown in FIG. 9A, if tissue within the right atrium 92 of the heart 90 is to be treated, the catheter 12 may be introduced through the venous system into the superior or inferior vena cava (superior approach being shown in FIG. 9A) and into the right atrium 92.

Turning to FIG. 9B, once within the right atrium 92, the balloon 50 may be expanded, and the apparatus 10 may be manipulated to place the distal surface 54 of the balloon 50 into contact with the wall 94 of the heart 90 within the right atrium 92. Optionally, this manipulation may involve steering the distal end 16 of the apparatus 50, e.g., using one or more pullwires or other steering mechanisms actuated from the proximal end (not shown) of the apparatus 10, as described elsewhere herein.

In addition or alternatively, other imaging systems may be used to monitor the apparatus 10 to facilitate introducing the apparatus 10 into the heart 90. For example, external imaging systems, such as fluoroscopy, ultrasound, magnetic resonance imaging (MRI), and the like, may provide feedback as to the location and/or relative position of the distal end 16 of the apparatus 12. The distal end 16 may include one or more markers, e.g., radiopaque bands and the like (not shown), that may facilitate such imaging. External imaging may ensure that the apparatus 10 is generally oriented towards a target tissue structure before optical images are acquired and/or the apparatus 10 is manipulated more precisely.

With the distal surface 54 of balloon 50 placed against the wall 94 of the heart 90, the imaging assembly 60 (not shown, see, e.g., FIG. 7) of the catheter 12 may be activated to image the wall 94. Sufficient distal force may be applied to the apparatus 10 to squeeze blood or other fluid from between the distal surface 54 and the wall 94, thereby clearing the field and facilitating imaging the wall 94. Optionally, a substantially transparent fluid, e.g., saline, may be delivered through the catheter 12 (e.g., through accessory lumen 20a, not shown) and the tubular extension 40 to further direct blood or other fluid away from the distal surface 54 of the balloon 50 or otherwise clear the field of view of the imaging assembly 60.

Using the imaging assembly 60 to directly visualize the wall 94, the apparatus 10 may be moved along the wall 94 until a target structure is within the field of view. For example, tissue that has undergone necrosis changes color compared to otherwise healthy tissue, while scar tissue may appear white and/or shiny compared with healthy tissue. In addition, areas around damaged tissue may become hyperemic with increased blood flow. Using the imaging assembly 60 on the catheter 12 to distinguish necrotic tissue from healthy tissue, e.g., using the indicators just identified, necrotic tissue along the wall 94 may be identified for treatment.

Once a target tissue region has been identified for treatment using the imaging assembly 60, the apparatus 10 may be moved further, e.g., until the target tissue region is centered in the field of view or otherwise oriented in a desired manner relative to the tubular extension 40. As shown in FIG. 9C, the distal end 74 of the needle 70 may then be advanced from the distal end 16 of the catheter 12 to puncture and enter at least partially into the target tissue region. The needle 70 may be carried within the catheter 12 while the catheter 12 is introduced with the distal end 74 retracted within the distal end 16 or the needle 70 may be advanced into the catheter 12 after the catheter 12 is introduced into the heart 90 or even after the target tissue region is identified.

If not already provided, a source of stem cells (not shown) may be coupled to the proximal end 72 of the needle 70, and stem cells may be injected through the needle 70 (or through a plurality of needles, not shown, each needle having one or more holes) into the target tissue region. Once sufficient stem cells are delivered, the needle 70 may be retracted back into the distal end 16 of the catheter 12. Optionally, one or more additional regions of necrotic tissue may be identified and stem cells injected therein. Once the desired one or more regions are treated, the balloon 50 may be collapsed, and the apparatus 10 removed from the patient's body.

In other embodiments, one or more additional therapeutic and/or diagnostic agents may be delivered into tissue in addition to or instead of stem cells, similar to the methods just described. In addition, the apparatus 10 may also be used for antegrade or retrograde infusion of one or more agents into other regions of the vasculature under direct visual guidance.

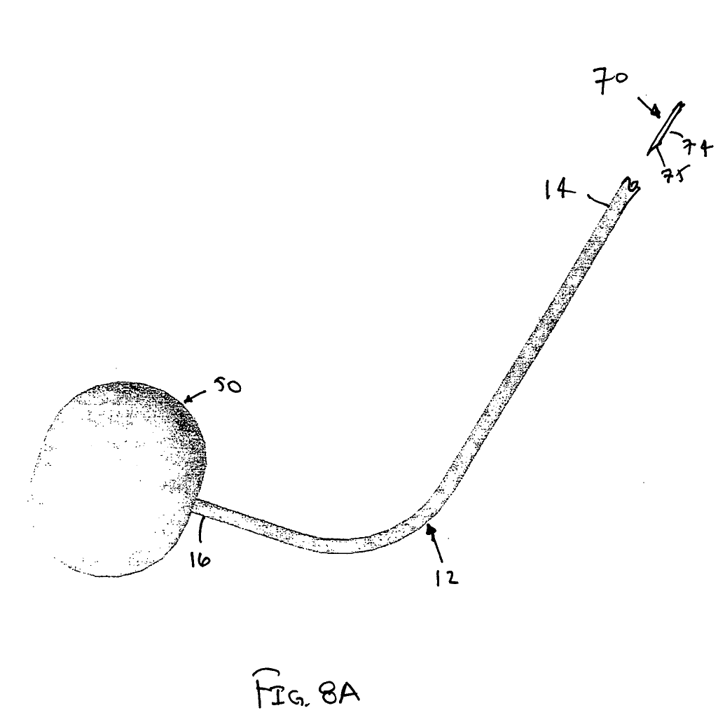

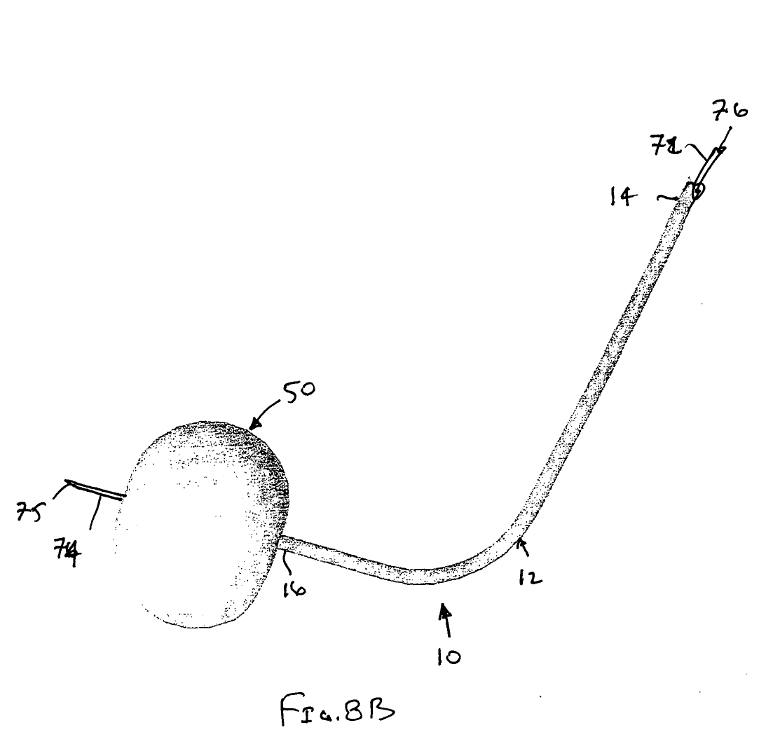

Turning to FIGS. 10A-10C, another method is shown for treating tissue within a patient's heart. In some procedures, it may be desirable to cross through a septal wall of a heart 90, e.g., the atrial septum 96, since the atria are relatively low pressure regions in the heart. For example, it may desirable to ablate or otherwise deliver electrical energy to tissue surrounding the pulmonary vein ostia 98 located within the left atrium 99, e.g., to treat atrial fibrillation, using access from the right side of the heart 90.

Similar to the previous embodiment, initially, the distal end 16 of the catheter 10 may be introduced into the right atrium 92 of the heart 90 with the balloon 50 collapsed (similar to FIG. 9A). Once the distal end 16 is located within the right atrium 92, the balloon 50 may be expanded, as shown in FIG. 10A, and the catheter 12 manipulated to place the distal surface 54 of the balloon 50 into contact with the atrial septum 96 of the heart 90 within the right atrium 92, as shown in FIG. 10B. Optionally, this manipulation may involve steering the distal end 16 of the apparatus 50, similar to the previous methods. Optionally, other imaging systems may be used to monitor the apparatus 10 to facilitate introducing the apparatus 10 into the heart 90 and/or ensure that the apparatus 10 is generally oriented towards the atrial septum 96 before optical images are acquired and/or the apparatus 10 is manipulated more precisely, also similar to the previous embodiments.

With the distal surface 54 of balloon 50 placed against the atrial septum 96 of the heart 90, the imaging assembly 60 may be activated to directly visualize the tissue of the septum 96. Sufficient distal force may be applied to the apparatus 10 to squeeze blood or other fluid from between the distal surface 54 and the septum 96, thereby clearing the field and facilitating imaging the septum 96. Optionally, a substantially transparent fluid, e.g., saline, may be delivered through the catheter 12 (e.g., through accessory lumen 20a, not shown) and the tubular extension 40 to further direct blood or other fluid away from the distal surface 54 of the balloon 50 or otherwise clear the field of view of the imaging assembly 60.

Using the imaging assembly 60 to image the atrial septum 96, the apparatus 10 may be moved along the wall 94 until a target structure is within the field of view. For example, in order to avoid puncturing the heart wall and/or to ensure that the left atrium 99 is accessed, a landmark or other target tissue structure, such as the fossa ovalis (“FOV”) 97, may be used to identify an appropriate location to puncture through the septum 96 into the left atrium 99.

Turning to FIG. 10C, once the fossa ovalis 97 (or other target tissue structure) has been identified and/or the apparatus 10 has been properly oriented relative to the fossa ovalis 97, the distal end 74 of the needle 70 may then be advanced from the catheter 12 to puncture the septum 96 and enter into the left atrium 99.

Turning to FIG. 10D, the balloon 50 may then be collapsed, and the distal end 16 of the catheter 12 may be advanced over the needle 70 through the septum 96 and into the left atrium 99. In this embodiment, the distal end 16 of the catheter 12 and/or the tubular extension 40 (if present) may be substantially tapered (not shown) or otherwise configured to facilitate advancing the catheter through the puncture in the septum 96. Once the distal end 16 of the catheter 12 is located within the left atrium 99, the needle 70 may be removed, and an energy probe 100 (or other probe for delivering electrical, light, thermal, or other energy) may be advanced through the accessory lumen 20a of the catheter 12 into the left atrium 99. The probe 100 may be used to ablate or otherwise treat tissue within the left atrium 99. For example, the probe 100 may include one or more electrodes (not shown) that may be used to ablate the ostium of one or more pulmonary veins 98, as is known in the art. A source or ablation energy, e.g., an electrical power generator (not shown), may be coupled to a proximal end of the probe 100, also as is known in the art.

Optionally, the balloon 50 on the catheter 12 may be expanded within the left atrium 99 and the imaging assembly 60 may be used to locate the pulmonary veins 98, using procedures similar to those described above. For example, the balloon 50 may be disposed over the pulmonary vein 98 being treated, whereupon the probe 100 may be advanced through the catheter 12 and the tubular extension 40 into the target ostium. The balloon 50 may remain expanded or may be collapsed when the probe 100 is activated to ablate the ostium of the pulmonary vein 98.

In an alternative embodiment, instead of advancing the catheter 12 into the left atrium 99 through the septum 96, a separate guide catheter (not shown) may be advanced over the needle 70 into the left atrium 99. The guide catheter may be advanced through the accessory lumen 20a of the catheter 12 or may be advanced over the entire catheter 12. The probe 100 may then be advanced through the guide catheter (e.g., after removing the needle 70) and manipulated to treat tissue within the left atrium 99.

In an alternative embodiment, the apparatus 10 may be used for visualizing the left atrial appendage before delivering an atrial closure device to close the left atrial appendage. For example, the apparatus 10 may be advanced through a puncture in the septum 98 to provide access during a procedure to reduce atrial appendage volume, e.g., using the probe 100. In other alternatives, the apparatus 10 may facilitate removing clots within the left atrium 99, and/or may be used to provide access to permit valve repair and/or replacement. In yet additional alternatives, the apparatus 10 may be used to directly visualize existing defects in a heart, such as atrial or ventricular septal defects. After using the apparatus 10 to identify and locate such defects, a guidewire (not shown) may be advanced through the catheter 12 and into or through the defect, which may facilitate repairing the defect, e.g., by delivering a closure device or otherwise closing the defect.

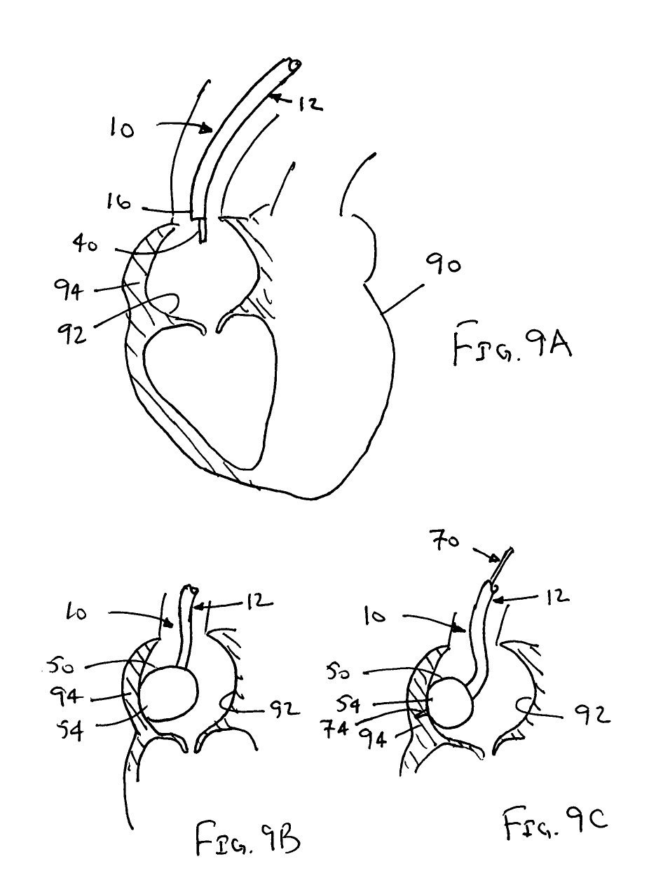

Turning to FIGS. 11A and 11B, in yet another embodiment, an apparatus 110 is shown that may facilitate advancing a guide catheter, energy probe, or other device through a puncture created in a septal wall, as described above. The apparatus 110 may include one or more components similar to the previous embodiments, e.g., a catheter 112 with a handle on a proximal end and a balloon and imaging assembly on a distal end thereof (not shown). Unlike the previous embodiments, the apparatus 110 may include an expandable lumen 120a for receiving an energy probe 100 or other device therethrough. Optionally, to further reduce the profile of the catheter 112, the catheter 112 may not have a pullwire and/or an accessory lumen, as described above with respect to previous embodiments of the catheter 12.

In an exemplary embodiment, the apparatus 110 may include a relatively thin-walled sheath 104 attached to or otherwise extending from an outer surface of the catheter 112. The sheath 104 may be formed from a substantially flexible and/or “floppy” material such that the sheath 104 defines the expandable lumen 120a, yet may be collapsed against or around the catheter 112, as shown in FIG. 11A. When the probe 100 or other device is advanced into the expandable lumen 120a, the sheath 104 partially separate from the catheter 12 and/or otherwise expand to accommodate receiving the device therethrough, as shown in FIG. 11B.

The sheath 104 may be expanded as the probe 100 or other device is inserted into the accessory lumen 120a at the proximal end of the apparatus 110 and is advanced towards the distal end. Alternatively, a fluid or other mechanism may be directed into the accessory lumen 120a to expand the sheath 104 before a device is inserted therein. Thus, the sheath 104 may be similar to the expandable sheaths described in co-pending application Ser. Nos. 10/433,321, filed Apr. 24, 2003, Ser. No. 10/934,082, filed Sep. 2, 2004, and Ser. No. 10/958,035, filed Oct. 4, 2004. The entire disclosures of these applications are expressly incorporated by reference herein.

The profile of the catheter 112 with the sheath 104 collapsed may be minimized, which may facilitate advancing the catheter 112 through a body lumen, over a needle (not shown), and/or through a puncture, e.g., in a septal wall, similar to the apparatus and methods described above. Once the catheter 112 is disposed through the puncture or septal wall, the probe 100 or other device (not shown), e.g., having a relatively large profile, may be advanced through the accessory lumen 120a of the sheath 104, rather than through a relatively small lumen in the catheter 112. The sheath 104 may facilitate passing the device through the puncture, e.g., dilating the puncture as necessary to accommodate receiving the device therethrough. Once the device is located in the second body cavity, the sheath 104 and/or catheter 112 may be removed from the patient's body, if desired, and the procedure completed similar to the previous embodiments.

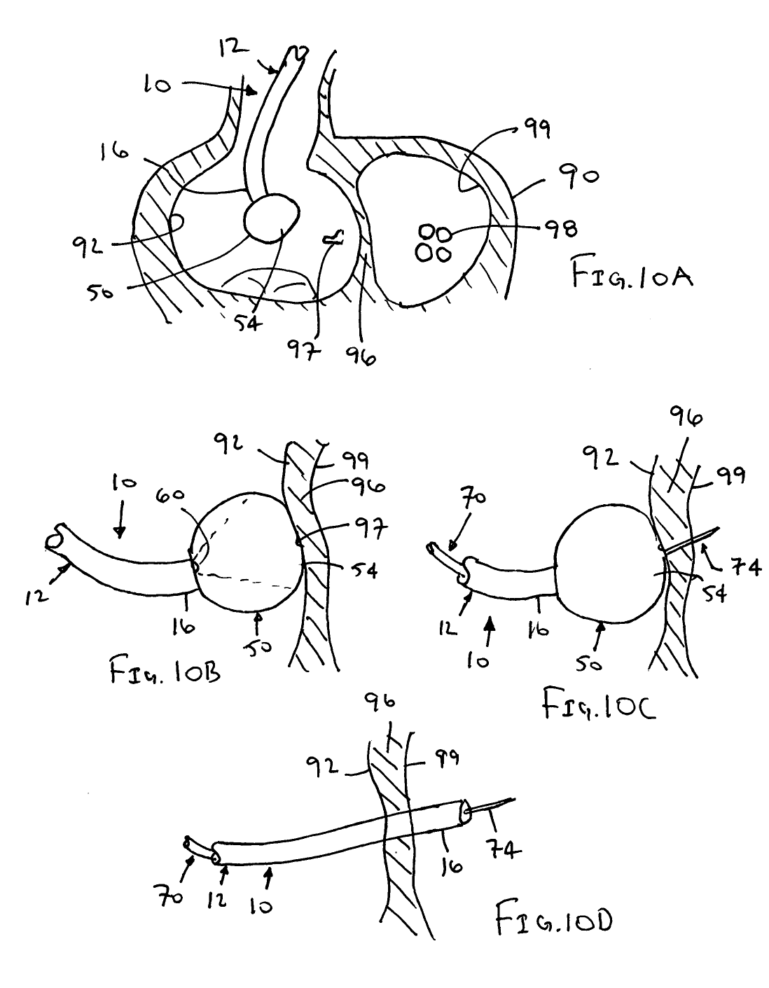

Turning to FIGS. 12A and 12B, an alternative embodiment of an apparatus 110′ is shown that includes an expandable sheath 104′ that may be collapsed to minimize a profile of the apparatus 110′ during delivery (as shown in FIG. 12A), and expanded to provide a relatively large accessory lumen 120a′ (as shown in FIG. 12B). Unlike the previous embodiments, the sheath 104′ may include a braided structure that may collapse to a relatively small cross-section. The braided structure may facilitate expansion and/or otherwise support the sheath 104′ during introduction and subsequent use.

The apparatus 110′ may include an optical imaging fiber 164′ and one or more illumination fibers 162′ (two shown), which may be embedded in or otherwise coupled to the sheath 104.′ Optionally, the sheath 104′ may include other components, e.g., one or more inflation lumens (not shown) that communicate with an interior of a balloon (also not shown) on a distal end of the apparatus 110.′ The illumination and imaging fibers 162,′ 164′ may be substantially fixed when the sheath 104′ is in the collapsed condition, thereby allowing tissue to be viewed beyond a distal end of the apparatus 110,′ similar to the previous embodiments.

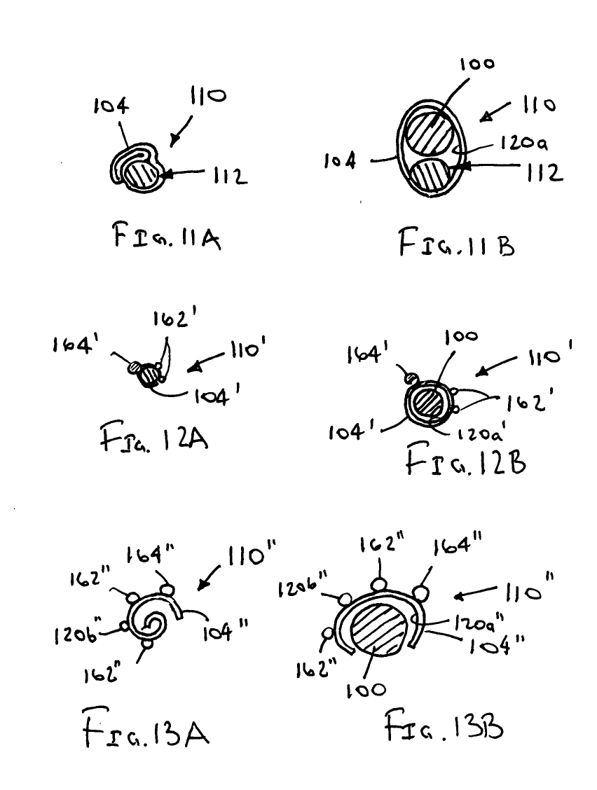

In a further alternative, the sheath 104′ may include a membrane, e.g., with or without braids, that may be expanded from the collapsed condition shown in FIG. 12A to an expanded condition shown in FIG. 12B. The lumens or components bonded or otherwise attached to the sheath 104′ may be embedded within or attached to an inner or outer surface of the membrane. In one embodiment, the membrane may be an elastomeric material, which may be elastically expandable to accommodate receiving the probe 100 or the device through the accessory lumen 120a.′ Turning to FIGS. 13A and 13B, yet another alternative embodiment of an apparatus 110″ is shown that includes a sheath 104″ carrying an optical imaging fiber 164,″ a pair of illumination fibers 162,″ and an inflation lumen 120b,″ which may be similar to the previous embodiments. Unlike the previous embodiments, the sheath 104″ may be a flat sheet coiled into an overlapping coil extending at least partially between the proximal and distal ends of the apparatus 110.″ For example, the sheath 104″ may be biased to a low profile configuration, e.g., the coiled configuration of FIG. 13A, yet may resiliently unroll to create a relatively large accessory lumen 120a″ for receiving an energy probe 100 or other device therein, similar to the previous embodiments.

The apparatus 110″ may be introduced into a patient's body in the low profile configuration shown in FIG. 13A. Once within a first body cavity, a balloon (not shown) on the distal end may be expanded, and an imaging assembly (also not shown) may be used to image tissue wall surrounding the first body cavity, e.g., to identify a location to puncture through the wall to a second body cavity, similar to the previous embodiments. Once the location is identified, a needle (not shown) may be advanced through the sheath 104,″ e.g., through the accessory lumen 120a or through another lumen (not shown) in the wall of the sheath 104.″ If the accessory lumen 120a is used, the sheath 104″ may unroll or otherwise expand partially to accommodate the needle.

The needle may be advanced from the sheath 104″ to puncture through the wall of the first body cavity and access the second body cavity. The apparatus 110″ may then be advanced over the needle through the puncture into the second body cavity with the balloon collapsed. Within the second body cavity, optionally, the balloon may be expanded again and used to image surrounding tissue to identify a target treatment site, similar to the previous embodiments.

With a target treatment site identified, the probe 100 or other device may be advanced through the accessory lumen 120a,″ as shown in FIG. 13B, e.g., after withdrawing the needle, and used to treat tissue at the target treatment site, similar to the previous embodiments. Upon completing the procedure, the probe 100 may be removed, whereupon the sheath 104″ may resiliently collapse again, facilitating its removal from the patient's body. Alternatively, the probe 100 or other device and apparatus 110″ may be removed substantially simultaneously or in other sequences.

It will be appreciated that elements or components shown with any embodiment herein are exemplary for the specific embodiment and may be used on or in combination with other embodiments disclosed herein.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. An apparatus for treating a condition within a patient's heart, comprising
a flexible tubular member comprising a proximal end, a distal end having a size and length for introduction into a chamber of a heart from an access location;
a substantially transparent expandable member carried by the distal end of the tubular member, the expandable member being expandable from a collapsed condition to an expanded condition, the expandable member being sufficiently conformable such that, in the expanded condition, the expandable member is directable against a tissue structure, thereby substantially displacing fluid between the expandable member and the tissue structure;
an optical imaging assembly carried by the distal end of the tubular member and at least partially surrounded by the expandable member, the optical imaging assembly for imaging the tissue structure beyond the distal end through the expandable member; and
one or more needles deployable substantially axially from the tubular member through a lumen in the expandable member for penetrating the tissue structure to treat tissue.

2. The apparatus of claim 1, further comprising a source of therapeutic agent coupled to the one or more needles, whereby the therapeutic agent may be delivered through the one or more needles into the tissue structure penetrated by the one or more needles.

3. The apparatus of claim 2, wherein the source of therapeutic agent comprises a source of stem cells.

4. The apparatus of claim 1, wherein the one or more needles have a length sufficient to penetrate through the tissue structure into a region beyond the tissue structure.

5. The apparatus of claim 4, further comprising a guide catheter advanceable over the one or more needles for accessing the region beyond the tissue structure penetrated by the one or more needles.

6. The apparatus of claim 5, wherein the guide catheter is advanceable over the tubular member after the expandable member is reduced to the collapsed condition.

7. The apparatus of claim 4, wherein the distal end of the tubular member is tapered such the tubular member may be advanced over the needle into the region beyond the tissue structure after the expandable member is reduced to the collapsed condition.

8. The apparatus of claim 4, further comprising an energy probe deployable through the tubular member for delivering electrical energy to tissue in the region beyond the tissue structure.

9. The apparatus of claim 8, further comprising an energy source coupled to the probe for delivering sufficient energy to the probe to ablate the tissue in the region beyond the tissue structure.

10. A method for delivering one or more therapeutic agents into tissue, comprising:
advancing a distal end of a tubular member through a body lumen into a body cavity;
expanding an expandable member on the distal end of the tubular member within the body cavity;
advancing the expanded expandable member against a wall of the body cavity;
imaging through the expandable member to observe tissue comprising a wall of the body cavity beyond the expandable member;
manipulating the tubular member to move the expandable member relative to the wall to identify a desired tissue structure; and
injecting one or more therapeutic agents from the tubular member into the desired tissue structure.

11. The method of claim 10, wherein the expandable member is advanced against the desired tissue structure before injecting the one or more therapeutic agents.

12. The method of claim 10, wherein the one or more therapeutic agents comprise stem cells.

13. The method of claim 10, wherein the desired tissue structure comprises infarcted tissue.

14. The method of claim 10, wherein the one or more therapeutic agents are injected into the desired tissue structure from a needle advanced from the tubular member into the desired tissue structure.

15. A method for treating tissue within a body lumen, comprising:
advancing a tubular member from a body lumen into a first body cavity;
expanding an expandable member on the distal end of the tubular member within the first body cavity;
advancing the expanded expandable member against a wall of the body cavity;
imaging through the expandable member to observe tissue comprising a wall of the body cavity beyond the expandable member;
manipulating the tubular member to move the expandable member relative to the wall to identify a desired tissue structure;
creating a puncture through the desired tissue structure into a second body cavity; and
performing a procedure within the second body cavity via the puncture.

16. The method of claim 15, wherein the step of performing a procedure, comprises:
collapsing the expandable member;
advancing the tubular member through the puncture into the second body cavity; and
expanding the expandable member in the second body cavity to image tissue surrounding the second body cavity;
manipulating the tubular member to identify a target tissue region surrounding the second body cavity; and
treating the target tissue region with a probe advanced through the tubular member.

17. The method of claim 16, wherein the step of treating the target tissue region comprises:
advancing an energy probe from the tubular member into contact with the target tissue region; and
delivering energy to the target tissue region to ablate the target tissue region.

18. The method of claim 16, wherein the puncture is created by advancing a needle from the tubular member through the desired tissue structure into the second body cavity.

19. The method of claim 18, wherein the step of performing a procedure within the second body cavity comprises:
advancing a guide catheter over the needle into the second body cavity; and
introducing a probe into the second body cavity via the guide catheter.

20. The method of claim 19, wherein the step of performing a procedure within the second body cavity further comprises delivering electrical energy from the probe to tissue within the second body cavity.
